(19) United States
(12) Patent Application Publication
(10) Pub. No.: US 2004/0267224 A1
(43) Pub. Date: Dec. 30, 2004

(54) Sanitary napkin with abbreviated side flaps

(76) Inventor: John T. Ulman, Woodbridge, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/603,250

(22) Filed: Jun. 25, 2003

(51) Int. Cl. A61F 13/15; A61F 13/20

(52) U.S. Cl. 604/385.04

(57) ABSTRACT

A sanitary napkin having a main body, the main body comprising a fluid-permeable cover layer, a liquid-impervious barrier layer and an absorbent system intermediate the cover layer and the barrier layer. The main body comprises a front portion, a rear portion and a center portion intermediate the front portion and the rear portion. The front portion extends laterally to at least one outermost front distal point and the rear portion extends laterally outward to at least one outermost rear distal point. A side flap projects laterally outward from the absorbent system and laterally outward from a proximal edge and is adapted to be folded over a crotch portion a user's undergarment. The side flap extends laterally outward from the proximal edge to a distal end. The distal end is not substantially laterally outward from the at least one outermost front distal point. Also, the distal end is not substantially laterally outward from the at least one outermost rear distal point. The at least one outermost front distal point, the at least one outermost rear distal point, and the side flap are each positioned on a single side of a longitudinally extending center line of the sanitary napkin.

### BACKGROUND OF THE INVENTION

1. Field of the Invention

The present invention generally relates to sanitary napkins, and more particularly to a sanitary napkin having side flaps.

2. Background of the Related Art

Sanitary napkins having side flaps are disclosed in the literature are commercially available. Generally, side flaps extend laterally from an absorbent system and are intended to fold over a crotch region of a wearer's panties. Thus the flaps are disposed between the wearer's panties in the crotch region and the wearer's thighs. The side flaps may be provided with attachment means such as adhesive in order to secure the napkin to the wearer's panties.

Side flaps generally perform two functions. First, the flaps prevent bodily exudates such as blood and menses from soiling the wearer's panties by forming a protective barrier to these exudates. Secondly, the flaps when affixed to the underside of the panties, the napkin is thereby stabilized with respect to the panties. The stabilizing effect is due to the fact that the napkin is prevented from undergoing significant shifts in position relative to the panties while the product is in use.

Unfortunately, the side flaps can be cumbersome for the wearer in that they require the wearer to perform the step of folding the wings around the undergarment when placing the napkin in the panties. Typically, the wearer is also required to remove release paper to expose adhesive in order to secure the side flaps to the panties. Adhesive on the side flaps also tends to make the sanitary napkin difficult to remove from the wearers undergarment. Furthermore, some users of sanitary napkins find side flaps to be uncomfortable in that the side flaps bunch up in use. Also, traditional sanitary napkins with flaps require additional material in order to form the flaps, thereby incurring additional manufacturing expense. Therefore, a need exists for a sanitary napkin that overcomes the above-mentioned drawbacks.

### SUMMARY OF THE INVENTION

In accordance with the present invention a sanitary napkin is provided. The sanitary napkin comprises a main body, the main body comprising a fluid-permeable cover layer, a liquid-impervious barrier layer and an absorbent system intermediate the cover layer and the barrier layer. The main body includes a front portion, a rear portion and a center portion intermediate the front portion and the rear portion. The front portion extends laterally to at least one outermost front distal point and the rear portion extends laterally outward to at least one outermost rear distal point.

A side flap projects laterally outward from the absorbent system, extending laterally outward from a proximal edge of the center portion and is adapted to be folded over a crotch portion a user's undergarment in use. The side flap extends laterally outward from the absorbent system along the proximal edge to a distal end. The distal end is not substantially laterally outward from the at least one outermost front distal point. Also, the distal end is not substantially laterally outward from the at least one outermost rear distal point. The at least one outermost front distal point, the at least one outermost rear distal point, and the side flap are each positioned on a single side of a longitudinally extending center line of the sanitary napkin.

### BRIEF DESCRIPTION OF THE DRAWINGS

A more particular description of the invention, briefly summarized above may be had by reference to the embodiments thereof that are illustrated in the appended drawings. It is to be so noted, however, that the appended drawings illustrate only typical embodiments of the invention and, therefore, are not to be considered limiting of its scope, for the invention may admit to other equally effective embodiments.

FIG. 1 is a top plan view of a sanitary napkin consistent with embodiments of the invention described herein;

FIG. 2 is an expanded, fragmented view of an anterior notch of FIG. 1;

FIG. 3 is an expanded, fragmented view of a posterior notch of FIG. 1;

FIG. 4 is a cross-sectional view of the sanitary napkin of FIG. 1 taken through line 3-3 of FIG. 1;

FIG. 5 is a cross-sectional view of an alternative embodiment of the sanitary napkin of the present invention, taken through line 3-3 of FIG. 1;

FIG. 6 is a partial perspective view of a sanitary napkin consistent with embodiments described herein shown attached to a wearer's undergarment;

FIG. 7 is an exploded view of region A of FIG. 7;

FIG. 8 is a bottom plan view of the sanitary napkin of FIG. 1; and

FIG. 9 is a bottom plan view of an alternative embodiment of the sanitary napkin of FIG. 1.

To facilitate understanding identical reference elements have been used, wherever possible, to designate identical elements that are common to the figures.

### DETAILED DESCRIPTION

The present invention generally relates to sanitary napkins adapted to be worn in a crotch region of a wearer's undergarment. As used herein, the term “sanitary napkin” refers to an article which is worn by females in an undergarment adjacent to the pudendal region and which is intended to absorb and contain various exudates which are discharged from the body (e.g., blood, menses, urine, and the like) and which is intended to be discarded after a single use (i.e., it is not intended to be laundered or otherwise restored or reused).

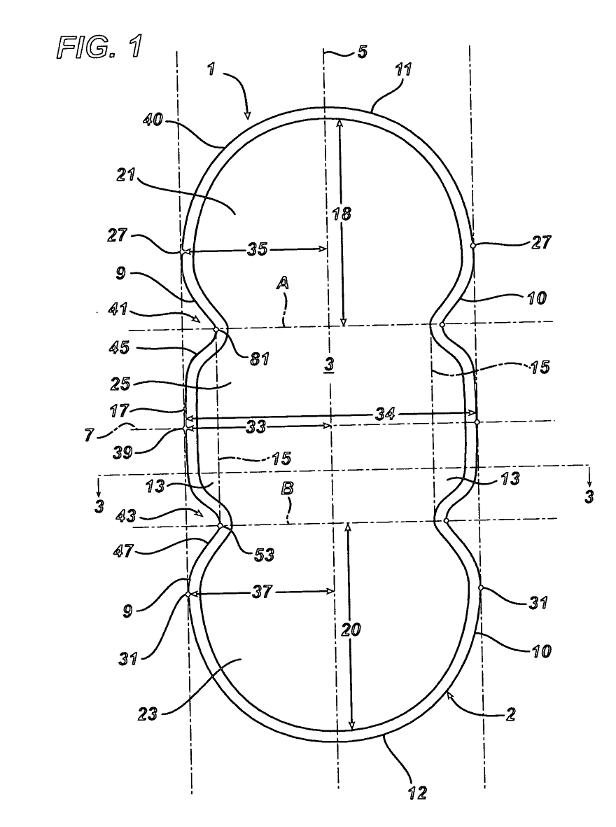

A specific implementation of a sanitary napkin 1, in accordance with embodiments of the invention described herein, is illustrated in FIG. 1. The sanitary napkin 1 has a footprint boundary 2 that, when viewed from above, as shown in FIG. 1, defines the spatial boundaries of the sanitary napkin 1. The sanitary napkin 1 is characterized as having an imaginary, longitudinally-extending centerline 5 and an imaginary transversely-extending centerline 7 that is generally perpendicular to the longitudinally-extending centerline 5 (the longitudinally-extending centerline 5 and the transversely-extending centerline 7 are shown in phantom in FIG. 1). The footprint boundary 2 generally consists of a first longitudinally-extending side edge 9, a second longitudinally-extending side edge 10 that is opposite the first longitudinally-extending side edge 9, a first transversely-extending end 11, and a second transversely-extending end 12 that is generally opposite the first transversely-extending end 11.

The sanitary napkin has a main body 3 that comprises a front portion 21, a rear portion 23, and a center portion 25 that is intermediate the front portion 21 and the rear portion 23. As shown in FIG. 1, the front portion 21 is bounded by an imaginary line A (shown in phantom in FIG. 1) that is perpendicular to the longitudinally-extending centerline 5 and extends from the first longitudinally-extending side edge 9 to the second longitudinally-extending side edge 10. The front portion 21 is also bounded by a portion of the footprint boundary 2. Similarly, the rear portion 23 is bounded by an imaginary line B (also shown in phantom in FIG. 1) that is perpendicular to the longitudinally-extending centerline 5 and extends from the first longitudinally-extending side edge 9 to the second longitudinally-extending side edge 10. The rear portion 23 is also bounded by a portion of the footprint boundary 2. The front portion 21 is adjacent to the center portion 25 and is separated from the center portion 25 by the imaginary line A. The rear portion 23 is adjacent to or juxtaposed with the center portion 25 and is separated from the center portion 25 by the imaginary line B. The center portion 25 is intermediate the front portion 21 and the rear portion 23 and is bounded by line A, line B, as well as a proximal edge 15 of the center portion 25, described below, on either side of the longitudinally-extending centerline 5.

The sanitary napkin 1 depicted in FIG. 1 has a front portion 21, a rear portion 23, and a center portion 25 intermediate the front portion 21 and the rear portion 23. An anterior notch boundary 45 generally defines the boundary line A that separates the front portion 21 of the main body 3 from the center portion 25 of the main body 3. Similarly, a posterior notch boundary 47 generally defines the boundary line B that separates the rear portion 23 of the main body 3 from the center portion 25 of the main body 3.

The notch boundaries 45, 47 have been formed to interrupt stresses on the sanitary napkin 1 that would, in a traditional sanitary napkin, be transmitted longitudinally throughout the sanitary napkin, compromising fit to a wearer's body. In use, the center portion 25 of the sanitary napkin 1 has a width 34 that is greater than a width of the narrowest crotch region of a user's undergarment thus permitting the flaps 13, 14 to fold around an edge of the undergarment. The front portion 21 of the sanitary napkin 1 is capable of bending around a wearer's pelvis and is generally adapted to cover and fit the body's anterior region of the mons pubis. In a traditional sanitary napkin, bending in the front of the napkin creates longitudinal creases that are typically transmitted through the length of the napkin. The center portion 25 of the sanitary napkin 1 is capable of interrupting these creases so that fit to the body is not compromised in the center portion 25.

The center portion 25 is also capable of deforming, such as by compression or bending, more than the front portion 21 and the rear portion 23, also allowing better fit to the wearer's labia majora and vestibule. 23. The rear of a traditional sanitary napkin is often forced into a flattened or “V” shape due its inability to move independently from the center of the napkin. The rear portion 23 of the sanitary napkin 1 is capable of independently deforming with respect to the center portion 25. As such, the sanitary napkin 1 is capable of fitting more closely to a posterior perineum and buttocks region of the wearer.

In one embodiment of the invention, the footprint boundary 2, are substantially symmetric about the longitudinally-extending centerline 5. For ease of exposition, the sanitary napkin 1 on one side of the longitudinally-extending centerline 5 will be described in detail. It is to be understood that, due to this symmetry across the longitudinally-extending centerline 5, similar spatial relationships may exist on the other side of the longitudinally-extending centerline 5.

Note that while the sanitary napkin 1 may be symmetric about the transversely-extending centerline 7, this is not required. For example, the front portion 21 may be have a length 18 that is smaller than a length 20 of the rear portion 23. Such a relationship may be advantageous for an article that requires high fluid absorbing capacity such as is common for so-called “overnight” sanitary napkins.

The front portion 21 extends laterally outward to an outermost front distal point 27. The outermost front distal point 27 is outermost in a sense that it is a point in the front portion 21 on the footprint boundary 2 that is furthest away from the longitudinally-extending centerline 5 (i.e. the most laterally outward from the longitudinally-extending centerline 5). While FIG. 1 depicts only one outermost front distal point 27, there may be more than one outermost front distal point 27 on a particular side of the longitudinally-extending centerline 5. For example, the first longitudinally-extending side edge 9 may have regions of significant linearity such that there are a plurality of points on one side of the longitudinally-extending centerline 5, each of which are equidistant from the longitudinally-extending center line 5.

Similarly, the rear portion 23 extends laterally outward to at least one outermost rear distal point 31. Similar to the one or more outermost front distal points 27, there may be more than one outermost rear distal point 31 on a particular side of the longitudinally-extending centerline 5.

A flap 13 adjoins the center portion 25 of the main body 3 and extends laterally outward (i.e., away from the longitudinally extending centerline 5) from the proximal edge 15 (shown in phantom in FIG. 1). The proximal edge 15 is a line of juncture that divides the main body 3 from the flap 13. In one embodiment of the invention, the proximal edge 15 is substantially coincident with an edge of the absorbent system (described below in the section entitled, “SANITARY NAPKIN CONSTRUCTION”).

The flap 13 extends from the proximal edge 15 to a distal edge 17 which is the most laterally outward portion of the longitudinally-extending side edge 9 of the flap 13. The distal edge 17 may be generally arcuate in nature (e.g. concave with respect to the longitudinally-extending centerline 5, as shown in FIG. 1) or may have sections that are substantially linear or convex with respect to the longitudinally-extending centerline 5.

The distal edge 17, and, more specifically, the at least one outermost distal point 39 on the distal edge 17 of the flap 13, is not substantially laterally outward from the outermost front distal point 27. Furthermore, the at least one outermost distal point 39 of the flap 13 is not substantially laterally outward from the outermost rear distal point 31. Furthermore, if there are multiple the outermost distal points 39 on the distal edge 17 of the flap 13, all such points satisfy the limitation of “not substantially laterally outward” stated above.

By “not substantially laterally outward” it is meant that the at least one outermost distal point 39 of the flap 13 has a distance 33 from the longitudinally-extending centerline 5 that is less than about 1.15 times that of a distance 35 between the outermost front distal point 27 and the longitudinally-extending centerline 5. Furthermore, the distance 33 of the distal edge 17 from the longitudinally-extending centerline 5 is less than about 1.15 times that of a distance 37 between the outermost rear distal point 27 and the longitudinally-extending centerline 5.

Thus, the distance 33 is not more than about 1.15 times that of either the distance 35 or the distance 37. The flaps 13, 14, as a result are considerably smaller than traditional flaps. It is an important feature of the invention that the center portion 25 be wide enough to cover a crotch portion of a user's underwear, in use. By allowing each flap 13 to extend slightly beyond the front portion 21 and the rear portion 23, the front portion 21 and the rear portion 23 may be made narrower and thus provide for a reduced cost of materials. The presence of the notch boundaries 45, 47 help provide independent motion for flaps 13 that extend slightly beyond points 27, 31. Despite this “abbreviation” of the flaps, the presence of the notch boundaries 45, 47 facilitates improvements in fit-to-body, thereby improving leakage performance.

In a further preferred embodiment, the distance 33 is not more than the distance 35 and the distance 33 is not more than the distance 37. Distances 33, 35, 37 are measured with the sanitary napkin 1 in an unfolded, flattened state, on a firm surface. This may be accomplished, for example, by placing 1 lb weights on side edges 9, 10, ends 11, 12 and on flaps 13, 14, in order to flatten the sanitary napkin 1 without appreciably disturbing or disrupting the structures therein.

As shown in FIG. 1, the relationships described above, specifically that the distal edge 17 is not substantially laterally outward from the either the outermost front distal point 27 or the outermost rear distal point 31 results in the flap 13 being abbreviated (i.e., short in comparison to prior art flaps). Furthermore, in a preferred embodiment, the flap 13 is, at least in part, structurally isolated from the front portion 21 and the rear portion 23 by an anterior notch 41 and a posterior notch 43, respectively. The structural isolation of the flap 13 from the front portion 21 allows the front portion 21 to move, at least in part, independently from the flap 13 and the center portion 25. Similarly, the structural isolation of the flap 13 from the rear portion 23 allows the rear portion 23 to move, at least in part, independently from the flap 13 and the center portion 25.

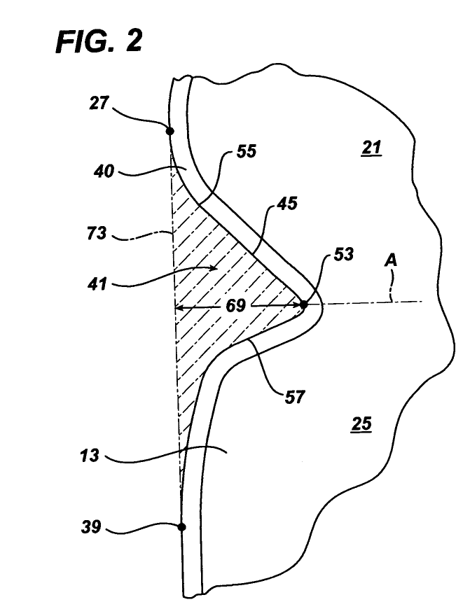

FIG. 2 shows an expanded, fragmented view of the anterior notch 41 of FIG. 1. In this embodiment of the invention, the anterior notch 41 has anterior notch boundary 45 that extends continuously from the outermost front distal point 27 to the outermost distal point 39 of the flap 13. Furthermore, the anterior notch boundary 45 includes portions that are laterally inward from the outermost front distal point 27. Similarly, the posterior notch 43 has posterior notch boundary 47 that extends continuously from the outermost rear distal point 31 to the outermost distal point 39. The posterior notch boundary 47 has a portion that is laterally inward from the at least one outermost rear distal points 31.

In the embodiment of the invention depicted in FIG. 2, the anterior notch 41 is longitudinally asymmetric. In other words, the anterior notch 41 is asymmetric with respect to the imaginary transversely-extending line A that is perpendicular to the longitudinally-extending centerline 5. The anterior notch boundary 45 includes a point of greatest inward extent 53. The imaginary line A separates a flat portion 55 (i.e., of slight curvature) of the notch boundary 45 and a steep portion 57 (i.e., of greater curvature than the flat portion 55) of the notch boundary 45 wherein the steep portion 57 is part of the footprint boundary 2 of the center portion 25 of the main body 3, and the flat portion 55 is part of the footprint boundary 2 of the front portion 21 of the main body 3. While the anterior notch 41 is described above as longitudinally asymmetric, this is not required. The anterior notch 41 may, in an alternative embodiment of the invention, be symmetric with respect to line A and therefore have no portion on either side of line A that are steeper or flatter with respect to one another.

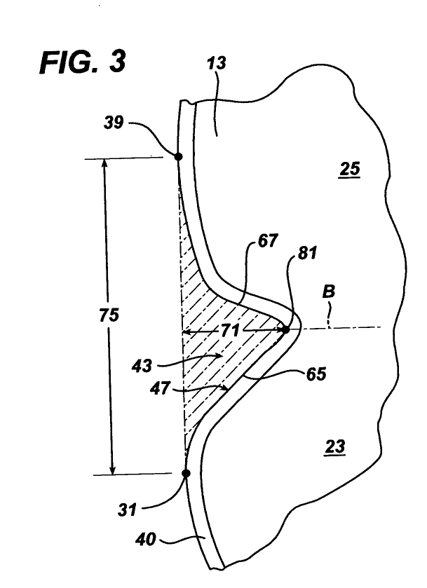

Similarly, as shown in FIG. 3, the posterior notch 43 may be asymmetrical in a manner similar to the anterior notch 41. In one embodiment of the invention, the posterior notch 43 has the imaginary line B that separates a flat portion 65 of the notch boundary 47 and a steep portion 67 of the notch boundary 47 wherein the steep portion 67 is part of the footprint boundary 2 of the center portion 25 of the main body 3 and the flat portion 65 is part of the footprint boundary 2 of the rear portion 23 of the main body 3.

Referring to FIGS. 2 and 3, the anterior notch 41 and the posterior notch 43 are characterized as having respective notch depths 69, 71. The notch depth 69 of the anterior notch is the distance, measured perpendicular to the longitudinally-extending centerline 5, between the point of greatest inward extent 53 and the outermost distal point 39. Similarly the notch depth 71 of the posterior notch 43 is the distance, measured perpendicular to the longitudinally-extending centerline 5, between a point of greatest inward extent 81 and the outermost distal point 39. The notch depth 69 and the notch depth 71 may be about the same in value or may be significantly different in value. The notch depths 69, 71 are preferably large enough to facilitate independent movement between the relevant portions 21, 23, 25 of the sanitary napkin 1 and preferably not too large such that the mechanical integrity (e.g., susceptibility to tearing) would be compromised. In one embodiment of the invention, the notch depths 69, 71 are each within a range of about 5 millimeters and about 20 millimeters. In another embodiment of the invention, the notch depths 69, 71 are each within a range of about 10 millimeters and about 12 millimeters.

The above-mentioned notch depths 69, 71 are especially suitable for a sanitary napkin 1 having a napkin width (the longest linear dimension of the footprint boundary 2 as measured parallel to the transversely-extending centerline 7) that is in a range from about 85 mm to about 110 mm. A ratio of notch depth to napkin width may be in a range from about 0.04 to about 0.24, or, in a more specific embodiment, from about 0.09 to about 0.14. These ratios are found to be advantageous to provide independent motion of the front portion 21, the center portion 23, and the rear portion 25 without resulting in a tendency of the sanitary napkin 1 to form tears or result in reduced coverage, in use.

Furthermore, the anterior notch 41 and the posterior notch 43 are characterized as having respective notch spans 73, 75. The notch span 73 of the anterior notch 41 is the distance, measured parallel to the longitudinally-extending centerline 5, between the outermost front distal point 27 and the outermost distal point 39 of the flap 13. Note that if there are multiple outermost distal point 39 of the flap 13 (because the distal edge 17 has significant regions of linearity or convexity), the distance is measured to the outermost distal point 39 that makes the measurement the least in value. Similarly the notch span 75 of the posterior notch 43 is the distance, measured parallel to the longitudinally-extending centerline 5, between the outermost rear distal point 27 and the outermost distal point 39 of the flap 13. The notch span 69 and the notch span 71 may be about the same in value or may be significantly different in value. The notch spans 73, 75 are preferably large enough to facilitate independent movement between the relevant portions 21, 23, 25 of the sanitary napkin 1 and preferably not too large such that the mechanical integrity of the sanitary napkin 1 would be compromised. In one preferred embodiment of the invention, the notch spans 73, 75 are within a range of about 45 millimeters and about 127 millimeters. In another embodiment of the invention, the notch spans 73, 75 are within a range of about 70 millimeters and about 95 millimeters. However, narrow notches, such as slits, and notches wider than the range described above are also contemplated.

### Sanitary Napkin Construction

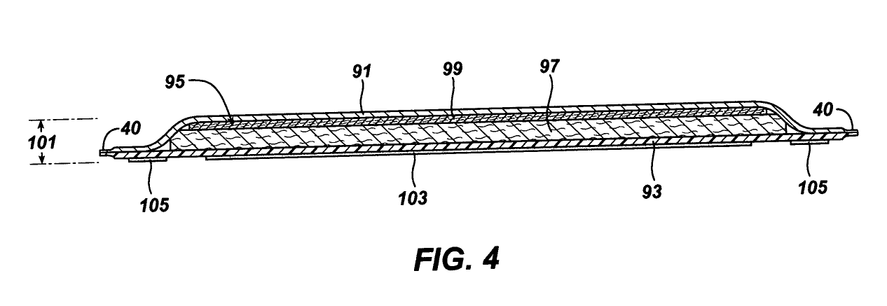

Referring to FIG. 4, the main body 3 of the sanitary napkin 1 generally includes a fluid-permeable cover layer 91, a liquid-impervious barrier layer 93 and an absorbent system 95 intermediate the fluid-permeable cover layer 91 and the liquid-impervious barrier layer 93.

The absorbent system 95 is confined to a longitudinally-extending center region, as shown in FIG. 4, such as a region substantially centered about the longitudinally-extending centerline 5, the transversely-extending centerline 7 or both (i.e., the flaps are free of absorbent material). The absorbent system 95 may comprise multiple material layers, such as an absorbent core 97 for retaining and storing fluid and a transfer (or acquisition) layer 38 for rapidly acquiring fluid from the cover layer 91.

The absorbent core 97 provides the means for absorbing menstrual fluid. The absorbent core 97 is generally compressible, comfortable and non-irritating to the user's skin. It can comprise any material used in the art for such purpose. Examples include comminuted wood pulp that is generally referred to as airfelt, creped cellulose wadding, absorbent foams, absorbent sponges, absorbent hydrogel materials, polymeric fibers, or any equivalent material or combinations of materials. The absorbent core 97 may be compressed such as by calendaring. The density of the absorbent core 97 may be in a range from about 0.1 g/cc to about 2.5 g/cc.

The transfer layer 99 provides the means of receiving body fluid from the fluid-pervious cover layer 91 and holding it until the absorbent core 97 has an opportunity to absorb it. The transfer layer 99 is, preferably, more dense than the fluid-pervious cover layer 91 and has a larger proportion of smaller pores than does the latter. These attributes allow the transfer layer 99 to contain body fluid and hold it away from the outer side of the fluid-pervious cover layer 91, thereby preventing the fluid from re-wetting the fluid-pervious cover layer 91 and its surface. However, the transfer layer 99 is preferably not so dense as to prevent the passage of the fluid through the transfer layer 99 and into the underlying absorbent core 97. The transfer layer 107 may comprise various material, including, for example, cellulose fibers such as from wood pulp, thermoplastic materials in fiber or other forms, polyester, rayon, organic binders, among other materials known to the art.

As shown in FIGS. 1 and 4, the cover layer 91 and the barrier layer 93 are joined at a seam 40 (also commonly referred to as a flange seal), around the entire periphery of the sanitary napkin 1. The purpose of this seam 40 is to unite the cover layer 91, barrier layer 93, and the absorbent system 95 of the sanitary napkin 1 into a unitary structure. The seam 40 can be formed by any means commonly used in the art for this purpose such as by gluing, crimping, or heat-sealing. Note that additional securement of the layers 91, 93, 97, 88 may be achieved by laminating one or more of these layers together. The notches 41, 43 are generally located in the seam 40.

The cover layer 91 is liquid permeable, and generally compliant, soft feeling, and non-irritating to the user's skin. It can be made from any of the materials conventional for this type of use. Non-limiting examples of suitable materials that can be used as the cover layer 91 are woven and nonwoven fabrics formed from polyester, polypropylene, nylon, and/or rayon fibers or the topsheet may be an apertured thermo-plastic film.

The barrier layer 93 is impervious to liquids and, thus, prevents menstrual fluid from soiling the clothing of the user. Any material used in the art for such purpose can be utilized herein. Suitable materials include embossed or non embossed polyethylene films and laminated tissue.

In one embodiment of the invention, the flap 13 is a continuous extension of the fluid-permeable cover layer 91 and the liquid-impervious barrier layer 93 of the main body 3. In this embodiment of the invention, no discemable change is evident from one side of the proximal edge 15 to the other side. In another embodiment of the invention, the flap 13 and the main body 3 are physically attached (such as by cutting and placing additional materials) along or near the proximal edge 15. The attachment may be via adhesive, heat seal, or other attachment means known in the art of sanitary napkin manufacture.

The sanitary napkin 1 has a thickness 101 that may be selected based upon desired technical properties of the sanitary napkin 1 (e.g. absorbency) or based upon consumer preference. In one embodiment of the invention, the thickness 101 is less than about 15 mm. In a further preferred embodiment, the thickness 101 is less than about 3 mm.

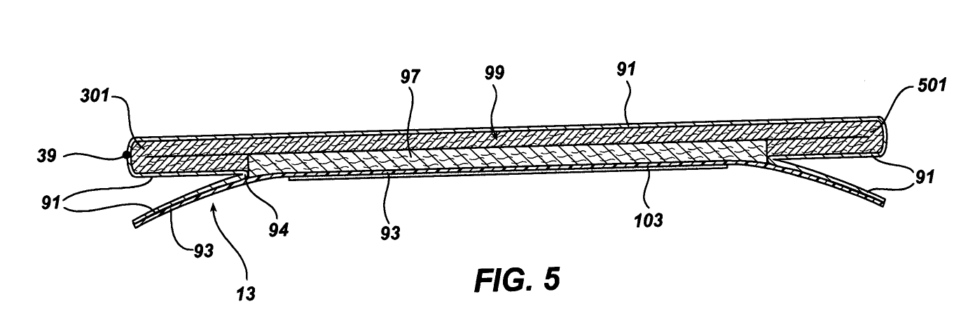

FIG. 5 depicts an alternative embodiment of the sanitary napkin 1 having an advantageous construction in which the cover layer 91 is folded around a resilient element 301 to form a cuff that creates an absorbent portion 501 that moves independently from the barrier layer 93 of the flap 13. The resilient element 301 is, in the embodiment depicted in FIG. 5, separated from the flap 13 along a hinge line 94 that permits independent rotational motion on the resilient element 301 from the flap 13. The resilient element 301 is preferably capable of being positioned close to the wearer's body, such as between the wearer's labia and inner thigh. The resilient element 301 is generally soft and compressible as well as resilient and serves to enhance comfort as well as protection from leakage. In order to achieve these enhancements, the resilient element 301 is preferably capable of being positioned close to the wearer's body, such as between the wearer's labia and inner thigh. The resilient element 301 generally comprises an absorbent material to facilitate absorption of fluid. The resilient element 301 may have a thickness greater than about 10 millimeters.

The resilient element 301 may comprise, for example, wood pulp, creped cellulose wadding, absorbent foams, absorbent sponges, absorbent hydrogel materials, polymeric fibers, thermoplastic materials in fiber or other forms, polyester, rayon, organic binders, among other materials, or combinations of such materials.

In this embodiment of the invention, the resilient element 301 may extend into or connect with the main body 3, as shown in FIG. 5. The resilient element 301 is shown as an extension of the transfer layer 95, and the transfer layer 95 is folded to provide additional loft and resilency, although the fold is not required. In an alternative embodiment of the invention, the transfer layer 95 and/or the absorbent core 97 may be selectively compressed in a central region of napkin 1 to provide relatively greater loft in the non-compressed lateral regions and thus provide a cuff with high loft and which will tend not to wick fluid from the more highly compressed central region of the napkin.

While FIG. 5 depicts the resilient element 301 as an extension of the transfer layer 95, the resilient element 301 may be an extension of the absorbent core 97 or both the transfer layer 95 and the absorbent core 97. Alternatively, the resilient element 301 may be a separate layer. In accordance with this latter embodiment, the resilient element 301 may be loftier and/or more voluminous, than the absorbent core 97 and transfer layer 99.

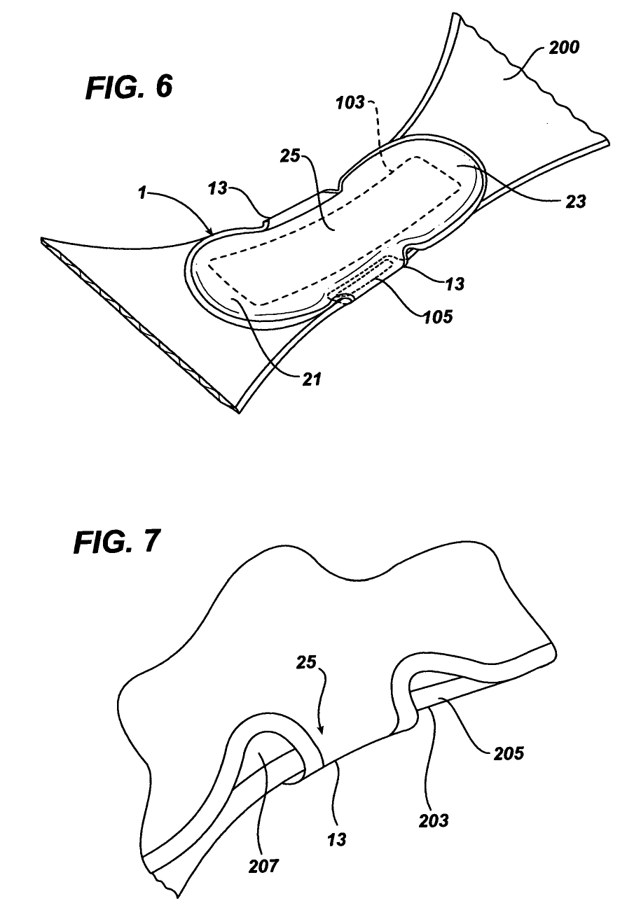

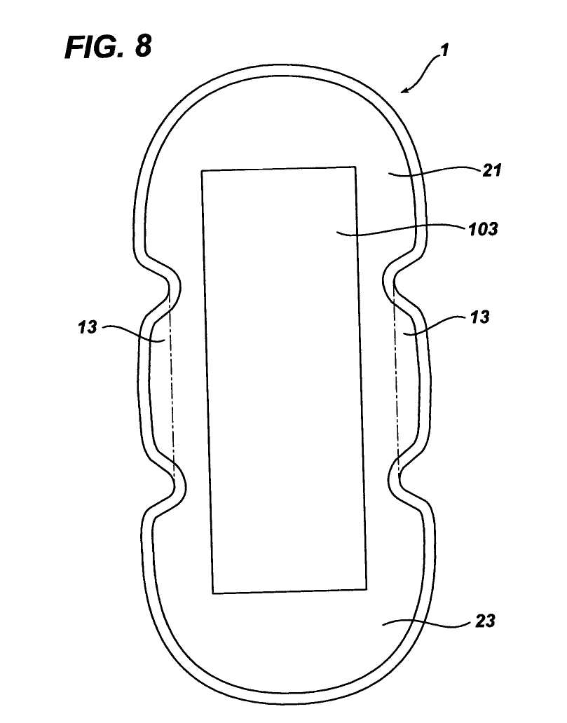

Referring to FIG. 6 the sanitary napkin 1 is placed in the crotch portion of an undergarment 200, with the front portion 21 positioned towards the anterior of the body with respect to the wearer and the rear portion 23 is positioned towards the posterior of the body. Referring to FIGS. 6, 7, 8 and 9, the sanitary napkin 1 generally includes a positioning adhesive such as a center positioning adhesive 103 to allow securement of the sanitary napkin 1 to the undergarment of the user. The center portion 25 of the sanitary napkin 1 is adapted to cover the narrow crotch portion of the undergarment 200, such that, in use, the flaps 13, 14 may each be folded over a side edge 205 of the crotch portion of the undergarment 200.

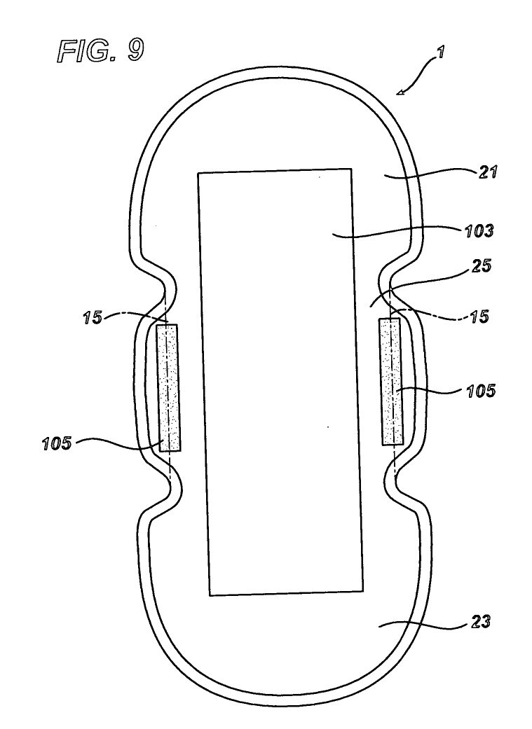

The flaps 13, 14 may be free of positioning adhesive. However, a flap positioning adhesive 105 may be optionally placed on the back of the side flaps 13, as shown in FIG. 9 (and in phantom in FIG. 6), to enhance the securement of the sanitary napkin 1 to the undergarment 201. In particular, for embodiments of the invention that include the absorbent portion 501 that moves independently from the barrier layer 93 of the flap 13 (as shown by example in FIG. 5), the additional securement from the flap positioning adhesive 105 does not detract from ability of the front portion 21, the center portion 25 and the rear portion 23 of the sanitary napkin 1 to move independently from one another.

The flap positioning adhesive 105 may be contained entirely within the flap 13 (i.e., laterally outward from the proximal edge 15) such that when the flap 13 is folded around a side edge of a wearer's undergarment 201, the flap positioning adhesive 103 preferably contacts an underside 203 of the wearer's undergarment 201. Alternatively, the flap positioning adhesive 105 may be partially within the flap 13 and partially within the center portion 25 of the main body 3. In this later embodiment, as shown in FIG. 9, when the flap 13 is folded over the side edge of the wearer's undergarment, the flap positioning adhesive 103 may, for example, contact an underside 203 as well as the side edge 205 as well as perhaps a top surface 207 of the undergarment 201.

The sanitary napkin 1 may be manufactured using process steps known to the art of sanitary napkin manufacture. The footprint boundary 2 may be formed by, for example, using a die roller having cutting blades in order to cut the desired shape (e.g. notches 41, 43) into the various material layers comprising the sanitary napkin 1.

Embodiments of the present invention are advantageous in that by having the distal edge 17 not substantially laterally outward from the either the outermost front distal point 27 or the outermost rear distal point 31, the front portion 21, the center portion 25, and the rear portion 23 of the sanitary napkin 1 can move independently from one another to achieve enhanced fit to the user's body. The notch boundaries 45, 47 enable improved performance via allowing independent motion of front portion 21, the center portion 25, and the back portion 23. Furthermore, the resilient element 301 enables improved performance by providing comfort as well as gasketing of bodily fluid. This is possible in an absorbent article with flaps 13 that would traditionally be considered as too small to provide adequate fit and protection.

Furthermore, the side flaps 13 provide protection to the user without the problems of bunching found in traditional flaps. In addition, the sanitary napkin 1 does not require flap positioning adhesive 105 and does not therefore require the user to suffer the inconvenience of remove release paper from the flaps 13. Furthermore, the optional resilient element 301 compliments the abbreviated flaps 13, 14 by achieving enhanced comfort and providing leakage protection that is comparable to sanitary napkins having traditional flaps. Also, because the side flaps 13, 14 are abbreviated, less materials are wasted in the manufacturing process.

While the foregoing is directed to various embodiments of the invention, other and further embodiments may be devised without departing from the basic scope thereof, and the scope thereof is determined by the claims that follow:

1. A sanitary napkin comprising:
a main body, the main body comprising a fluid-permeable cover layer, a liquid-impervious barrier layer and an absorbent system intermediate the cover layer and the barrier layer, wherein the main body comprises a front portion, a rear portion and a center portion intermediate the front portion and the rear portion, and wherein the front portion extends laterally to at least one outermost front distal point and the rear portion extends laterally outward to at least one outermost rear distal point; and
a side flap projecting laterally outward from the absorbent system, extending laterally outward from a proximal edge of the center portion and adapted to be folded over a crotch portion a user's undergarment in use, the side flap extending laterally outward from the proximal edge to a distal end, wherein the distal end is not substantially laterally outward from the at least one outermost front distal point and the distal end is not substantially laterally outward from the at least one outermost rear distal point, and wherein the at least one outermost front distal point, the at least one outermost rear distal point, and the side flap are each positioned on a single side of a longitudinally extending center line of the sanitary napkin.

2. The sanitary napkin of claim 1 wherein one or more outermost distal points of the flap have a distance from the longitudinally-extending centerline that is less than a distance between the one or more outermost front distal points and the longitudinally-extending centerline, and wherein the one or more outermost distal points of the flap have a distance from the longitudinally-extending centerline that is less than a distance between the outermost rear distal points and the longitudinally-extending centerline.

3. The sanitary napkin of claim 1 wherein the flap is, at least in part, structurally isolated from the front portion by an anterior notch, and the flap is, at least in part, structurally isolated from the rear portion by a posterior notch.

4. The sanitary napkin of claim 1 wherein the side flap projects outward to at least one outermost distal point of the flap, and wherein the sanitary napkin further comprises an anterior notch defined by an anterior notch boundary that extends continuously from the at least one outermost front distal points to the at least one outermost distal points of the flap, and wherein the at least one outermost distal points of the flap are located on the single side of the longitudinally-extending centerline.

5. The sanitary napkin of claim 1 wherein the anterior notch boundary includes portions that are laterally inward from the outermost front distal point.

6 The sanitary napkin of claim 1 wherein the flap is free of positioning adhesive.

7. The sanitary napkin of claim 1 wherein the flap includes a flap positioning adhesive on a bottom surface the liquid-impervious barrier layer.

8. The sanitary napkin of claim 3 wherein a notch selected from the group consisting of the anterior notch, the posterior notch, and combinations thereof is longitudinally asymmetric.

9. The sanitary napkin of claim 3 wherein the notch has notch depth in a range from about 11 mm to about 20 mm.

10. The sanitary napkin of claim 3 wherein the notch has a notch span in a range from about 70 mm to about 127 mm.

11. The sanitary napkin of claim 1 wherein the fluid-permeable cover layer and the liquid-impervious barrier layer each extend homogeneously across the proximal edge from the main body to the flap.

12. The sanitary napkin of claim 1 further comprising a resilient element.

13. The sanitary napkin of claim 12 wherein the resilient element is separated from the flap along a hinge line.

14. A sanitary napkin comprising:
a main body, the main body comprising a fluid-permeable cover layer, a liquid-impervious barrier layer and an absorbent system intermediate the cover layer and the barrier layer, wherein the main body comprises a front portion, a rear portion and a center portion intermediate the front portion and the rear portion, and wherein the front portion extends laterally to at least one outermost front distal point and the rear portion extends laterally outward to at least one outermost rear distal point; and
a side flap projecting laterally outward from the absorbent system and laterally outward from a proximal edge of the center portion and adapted to be folded over a crotch portion a user's undergarment, wherein the side flap extends laterally outward from the proximal edge to an outermost distal point of the flap, wherein the outermost distal point of the flap is not laterally outward from the at least one outermost front distal point and the outermost distal point of the flap is not laterally outward from the at least one outermost rear distal point, and wherein the at least one outermost front distal point, the at least one outermost rear distal point, and the side flap are each positioned on a single side of a longitudinally extending center line of the sanitary napkin, and wherein the flap is, at least in part, structurally isolated from the front portion by an anterior notch, and the flap is, at least in part, structurally isolated from the rear portion by a posterior notch.
